(19) United States
(12) Patent Application Publication
(10) Pub. No.: US 2014/0287438 A1
(43) Pub. Date: Sep. 25, 2014

(54) Rapid and Inexpensive Assay for Evaluation of Antibody Efficacy with Custom-Designed Fluorescent Nanoparticles

(71) Applicant: South Dakota Board of Regents, Pierre, SD (US)

(72) Inventors: Victor Huber, Vermillion, SD (US); Grigoriy Sereda, Vermillion, SD (US); James Dale, Memphis, TN (US)

(73) Assignee: South Dakota Board of Regents, Pierre, SD (US)

(21) Appl. No.: 14/214,750

(22) Filed: Mar. 15, 2014

Related U.S. Application Data
(60) Provisional application No. 61/789,791, filed on Mar. 15, 2013.

(51) Int. Cl. G01N 33/569

(52) U.S. Cl. CPC G01N 33/56972
USPC 435/7.24

(57) ABSTRACT

A method for determining the efficacy of a vaccine comprising: providing serum from an animal inoculated with a vaccine; providing a plurality of antigen-linked nanoparticles; contacting the serum with the plurality of antigen linked nanoparticles; contacting the serum and the plurality of antigen linked nanoparticles with a plurality of Fc receptor-expressing cells; measuring amount antigen-linked nanoparticle uptake by of the Fc receptor-expressing cells; determining efficacy of the vaccine by comparing the level of antigen-linked nanoparticle uptake to a baseline level of uptake wherein a greater nanoparticle uptake compared to the baseline level of uptake is indicative of greater vaccine efficacy.

### CROSS-REFERENCE TO RELATED APPLICATION(S)

This application claims priority from U.S. Provisional Application Ser. No. 61/789,791 filed Mar. 15, 2013.

### FIELD OF THE INVENTION

The field of the various inventions disclosed herein relates to assays to determine response to a vaccine. More specifically, the inventions relate to assays that recapitulate antibody interactions with host cells, as they would occur in vivo.

### BACKGROUND OF THE INVENTION

Traditional laboratory-based assays that measure the quantity of antibody are the current standard for determining vaccine efficiency. These quantity-based immune assays are frequently imperfect and are not typically designed to assign a host effector function for removal of the pathogen from the host. Current assays used to detect vaccine-induced antibodies to the influenza virus include Hemagglutination Inhibition (HAI), microneutralization, and ELISA. A major limitation of these assays is that they frequently are unable to differentiate between IgG isotypes, lack standardized reagents, and require handling of dangerous infectious materials. Further, they do not consider the contribution of Fc:Fc receptor interactions in their evaluation.

There is a need in the art for a safe, inexpensive, and simple assay to monitor these interactions. Such an assay would significantly improve diagnostic evaluation of vaccine-induced immunity against viral and other pathogens.

### BRIEF SUMMARY

Disclosed herein is a method for determining the efficacy of a vaccine comprising: providing serum from an animal inoculated with a vaccine; providing a plurality of antigen-linked nanoparticles; contacting the serum with the plurality of antigen linked nanoparticles; contacting the serum and the plurality of antigen linked nanoparticles with a plurality of Fc receptor-expressing cells; measuring amount antigen-linked nanoparticle uptake by of the Fc receptor-expressing cells; determining efficacy of the vaccine by comparing the level of antigen-linked nanoparticle uptake to a baseline level of uptake wherein a greater nanoparticle uptake compared to the baseline level of uptake is indicative of greater vaccine efficacy.

Disclosed herein is method for determining the immunogenic effect of a vaccine on a subject comprising: obtain serum from the subject prior to a vaccination; obtaining serum the subject after a vaccination; contacting the pre-vaccination serum and post-vaccination serum with a plurality of antigen-linked nanoparticles, contacting the plurality of antigen-linked nanoparticles with a plurality of Fc receptor-expressing cells; measuring the amount of antigen-linked nanoparticle uptake by the Fc receptor-expressing cells; and determining the effect of the immunogenic effect of vaccine on the subject by comparing the amount of antigen-linked nanoparticle uptake induced by pre-vaccination serum with post vaccination serum wherein a greater immunogenic effect is indicated by a greater level of uptake by post-vaccination serum.

While multiple embodiments are disclosed, still other embodiments will become apparent to those skilled in the art from the following detailed description, which shows and describes illustrative embodiments of the invention. As will be realized, the embodiments disclosed herein are capable of modifications in various obvious aspects, all without departing from the spirit and scope of the various inventions. Accordingly, the drawings and detailed description are to be regarded as illustrative in nature and not restrictive.

### BRIEF DESCRIPTION OF THE DRAWINGS

FIG. 1 shows an image of Fluorescein-doped silica nanoparticles (˜100 nm in diameter).

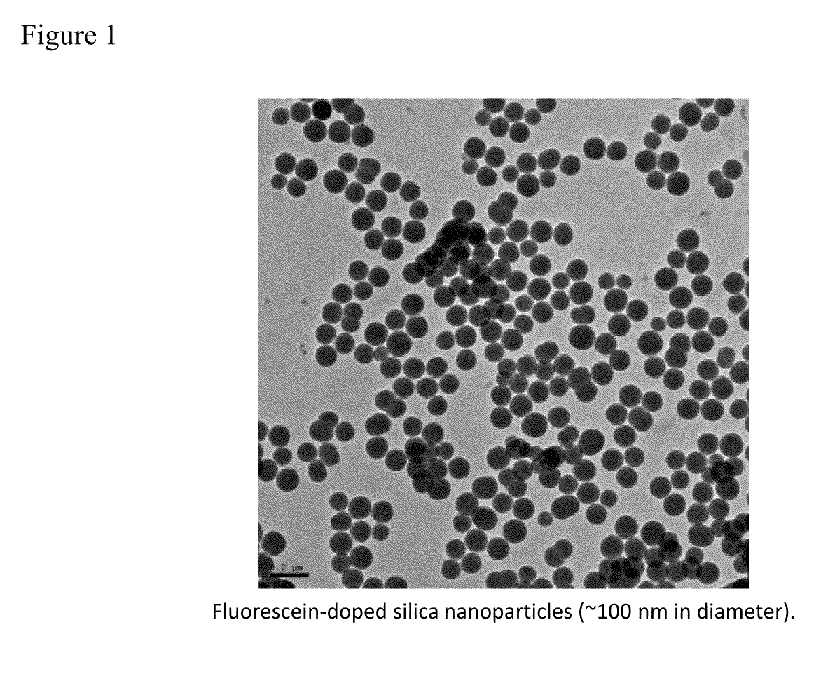

FIG. 2 shows a schematic of nanoparticles linked with the protein of interest at an optimized protein:nanoparticle ratio according to certain embodiments.

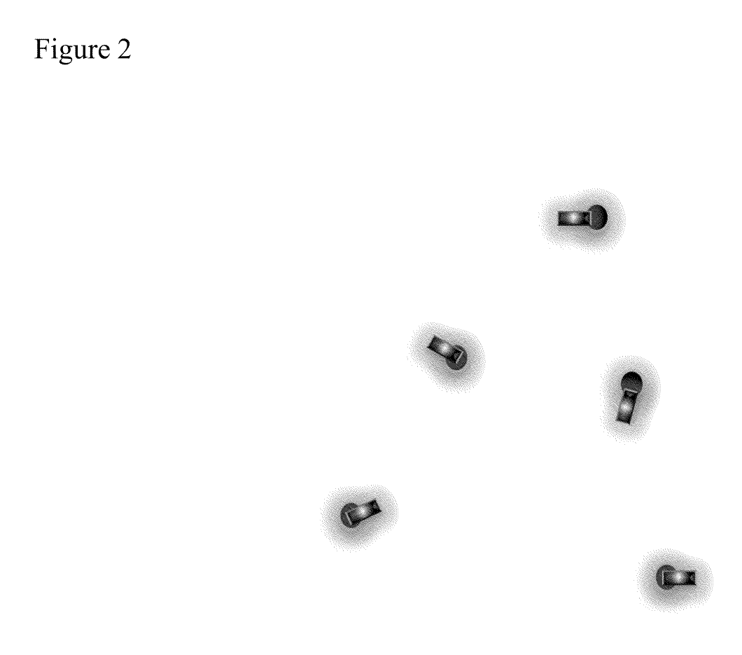

FIG. 3 shows a schematic antigen-linked nanoparticle-protein complex binding antibodies to the protein of interest according to certain embodiments.

FIG. 4 shows a schematic representation of binding and uptake of the nanoparticle-antigen-antibody complex by an Fc receptor-expressing cell according to certain embodiments.

FIG. 5 (A) shows a schematic representation of binding and uptake of the nanoparticle-antigen-antibody complex by an Fc receptor-expressing cell according to certain embodiments. (B) Shows a schematic of the assay method according to certain embodiments.

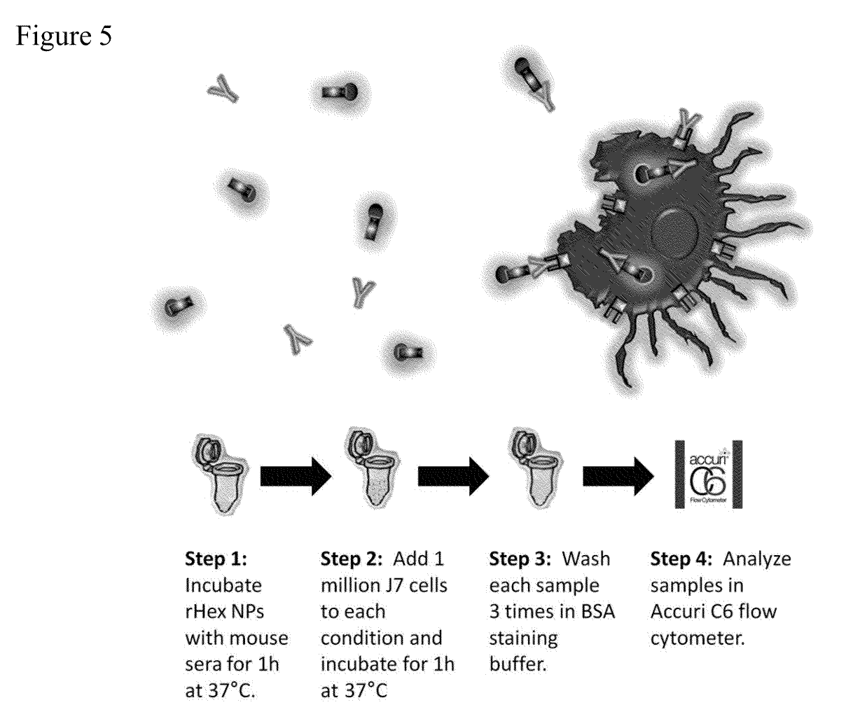

FIG. 6 shows confocal microscopy images demonstrating uptake of fluorescent nanoparticles by macrophages with either no serum (A), serum from unvaccinated animals (B), or serum from vaccinated animals (C).

FIG. 7 shows flow cytometry data quantifying macrophage uptake of fluorescent nanoparticles (A) demonstrates individual fluorescence peaks for a single representative from each group. (B) demonstrates the fluorescence units for multiple serum samples within each group.

FIG. 8 shows data of serum reactivity toward the influenza virus CA09 HA using HAI.

FIG. 9 shows HAI data for a variety of virus isolates.

FIG. 10 shows flow cytometery data showing specificity of antigen-linked nanoparticle uptake.

### DETAILED DESCRIPTION

Antibodies have evolved to interact with multiple pathogens through their variable, antigen-binding region (known as the Fab portion), while simultaneously interacting with host cells to actively clear the bound pathogen (using their constant, or Fc, region). The Fc region of an antibody interacts with receptors on host cells, known as Fc receptors, which can lead to uptake and killing of a pathogen. To date, the majority of antibody detection assays focus on interactions that occur at the Fab portion, with little attention paid to the Fc interactions that mediate clearance in vivo.

Current assays used to detect vaccine-induced antibodies to the influenza virus include Hemagglutination Inhibition (HAI), microneutralization, and ELISA. The HAI test, which is the least sensitive, detects antibodies that prevent binding of virus to red blood cells (RBCs). The test cannot differentiate antibody isotypes (IgG1 v. IgG2a). The defined 50% correlate of protection is a HAI antibody titer of 1:40. Other problems with the HAI test include the fact that the source of the virus is either infectious or inactivated and that there may be significant user error in the dilution of virus, dilution of red blood cells (0.5%) and reading of the positive/negative wells. Further, both historical and recent evidence demonstrates that the HAI titer cannot be used as the sole correlate of protection against influenza virus. For example, in studies that evaluated vaccine-induced immunity toward the highly pathogenic avian influenza virus (influenza A, H5N1 subtype), protection could be observed in animals with suboptimal HAI titers (<1:40). In the event of an H5N1 influenza virus pandemic, a more accurate correlate of immunity to demonstrate that a vaccinated individual would be protected against this virus would be critical. Furthermore, this correlate of immunity would need to be measured while meeting the defined biosafety handling criteria, which currently limit the use of H5N1 viruses and their by-products.

Another assay currently used is microneutralization, which detects antibodies that neutralize virus infection of MDCK cells. Similar to HAI, this test cannot differentiate antibody isotypes (IgG1 v. IgG2a) and it requires infectious virus. Errors in dilution of the virus are also common.

ELISA, the most sensitive test used, detects antibodies that bind virus particles (neutralizing and non-neutralizing). This test can differentiate antibody isotypes induced, but cannot assign effector function to these antibodies. Other problems with ELISA include the fact that standardized reagents are not available, a protective titer has not been defined (but is typically higher than HAI) and the test indicates presence but not function of antibody.

A major limitation to current lab-based assays used to measure correlates of immunity is that the majority of these assays, including ELISA, HAI, or neutralizing antibody assays, do not consider the contribution of Fc:Fc receptor interactions in their evaluation. Studies have shown that the presence of Fc receptors and Fc receptor-interacting antibodies contribute to clearance of an influenza virus infection, even when titers within HAI and neutralization assays demonstrate low levels of antibody present. The methods disclosed herein provide a safe, inexpensive, and simple assay to monitor these interactions to significantly improve diagnostic evaluation of vaccine-induced immunity against a wide array of pathogens.

In certain aspects, provided is a method for determining the efficacy of a vaccine, the method comprising: providing serum from an animal inoculated with a vaccine; providing a plurality of antigen-linked nanoparticles; contacting the serum with the plurality of antigen linked nanoparticles; contacting the serum and the plurality of antigen linked nanoparticles with a plurality of Fc receptor-expressing cells; measuring amount antigen-linked nanoparticle uptake by of the Fc receptor-expressing cells; determining efficacy of the vaccine by comparing the level of antigen-linked nanoparticle uptake to a baseline level of uptake wherein a greater nanoparticle uptake compared to the baseline level of uptake is indicative of greater vaccine efficacy.

In certain aspects vaccine efficacy is determined by assessing host effector function. According to further aspects, the method determines vaccine efficacy by assessing the vaccine's ability to trigger protective immunity. In still further aspects, vaccine efficacy is determined by the vaccine's ability to trigger pathogen clearing. In further aspects, the method determines vaccine efficacy by assessing the vaccine's ability to induce antibody-dependent cellular cytotoxicity. In yet further aspects, the method determines vaccine efficacy by assessing the vaccine's ability to induce antibody-dependant opsonophagocytosis. In further aspects a vaccine's efficacy is determined by its ability to produce antibodies that trigger Fc-receptor-dependent uptake. According to yet further aspects, the method's determination of vaccine efficacy does not rely on quantification of antibody production. That is, a vaccine may be deemed effective despite antibody production being relatively low if the antibodies produced facilitate Fc-receptor-dependant uptake. Conversely, a vaccine may be determined to be ineffective despite antibody production being relatively high if the antibodies produced do not facilitate Fc-receptor dependant uptake.

In certain embodiments, the methods disclosed herein relate to determining the efficacy of a preclinical vaccine. In further embodiments, the methods relate to comparing the efficacy of two or more vaccines in clinical use. In still further embodiments, the method relates to determining the effect of a vaccine on a subject. For example, once a subject has been vaccinated, the method disclosed herein is used to determine whether the vaccine has generated the desired immunogenic effect.

### Vaccine Types

The methods disclosed herein are used to determine the efficacy of a vaccine for any pathogen for which vaccination is effective. According to certain embodiments, the pathogen is a virus. In further embodiments, the virus is influenza. In still further embodiments, the pathogen is a bacterium. In yet further embodiments, the pathogen is a fungus. In certain aspects the vaccine is an attenuated live or killed vaccine, a subunit vaccine, a synthetic vaccine, or a genetically engineered vaccine. In still further embodiments, the vaccine is a toxoid vaccine.

In certain aspects the method relates to providing serum from an animal inoculated with a vaccine. In further aspects, purified antibodies from the serum of an animal inoculated with a vaccine are provided. In yet further aspects, monoclonal antibodies derived from immunized animals or developed in vitro are provided.

In certain aspects, the vaccinated animal is a mammal, fish or bird. In a yet further aspect, the mammal is a primate. In a still further aspect, the mammal is a human.

In certain aspects the animal is a domesticated animal. In a yet further aspect, the domesticated animal is poultry. In an even further aspect, the poultry is selected from chicken, turkey, duck, and goose. In a still further aspect, the domesticated animal is livestock. In a yet further aspect, the livestock animal is selected from pig, cow, horse, goat, bison, and sheep.

In certain embodiments, the animal is a laboratory animal. In further embodiments, the laboratory animal is a mouse, rat, gerbil, hamster, rabbit, ferret, or a primate.

### Antigen-Linked Nanoparticle

In certain aspects the invention relates to providing an antigen-linked nanoparticle. According to certain embodiments the antigen-linked nanoparticle serves to present a pathogen-associated antigen for binding by vaccine induced antibodies and a means for identifying/quantifying cells that uptake the antigen-linked nanoparticle.

### Antigen

According to certain embodiments, the antigen of the antigen-linked nanoparticle is associated with the pathogen against which the vaccine is directed. In certain embodiments, the antigen is hemagglutinin (HA). In further embodiments, the antigen is the influenza virus ectodomain of the M2 ion channel (M2e). In further embodiments, the antigen is a viral neuraminidase (NA). In addition to the listed proteins from influenza virus, antigens include other influenza virus-associated molecules, as well as molecules from viruses, bacteria, fungi, and other parasites that are associated with Fc receptor-mediated effector responses for host:pathogen interactions. In certain aspects the antigen is a recombinant protein, recombinant peptide, native protein or native peptide, chemically synthesized peptide, native carbohydrate or chemically synthesized carbohydrate. In certain embodiments, the antigen is noninfectious. The use of non-infectious antigens allows for evaluation of immune responses to vaccines against dangerous pathogens (like avian (H5N1) influenza A viruses or smallpox viruses) without needing to handle materials that are classified at biosafety level 3 or above (including HHS select agents and toxins) as is frequently required with many prior art assays.

### Nanoparticles

In certain aspects, the nanoparticle of the antigen-linked nanoparticle is configured to be linkable to an antigen of interests, to be capable of uptake by an Fc receptor-expressing cell, and to be detectable upon uptake. The composition of the nanoparticle can vary. According to certain embodiments, the nanoparticle is comprised of silicate. According to certain embodiments, nanoparticle compositions include but are not limited to metal oxides: SiO2, ZnO, Al2O3, CrO, SnO2 and TiO2; polymer nanoparticles including but not limited to polystyrene, Poly(d,1-lactic-co-glycolic acid) (PLGA), poly(ethylene glycol)-block-poly(aspartic acid) (PEG-PAA)-coated calcium phosphate; polyethylene glycol (PEG) covered or PEGylated nanoparticles; poly-vinyl-chloride (PVC); lipids and lipoproteins; proteins condensed nanoparticles comprising albumin and oligonucleotides; nanoparticles containing DNA in addition to inorganic molecules or non-nucleic acid polymers: polyethylene glycol/DNA nanoparticles, poly(methyl methacrylate)/poly(ethyleneimine)-nanoparticle/pDNA complexes, poly-1-Lysine-DNA complexes; fluorescent polymer nanoparticles; semiconductor nanoparticles for example, quantum dots, and other semiconductors.

In certain aspects, the antigen-linked nanoparticle is detectable. In certain embodiments, the nanoparticle is detectable by way of a florescent signal. In certain embodiments the florescent signal is generated by way of a reported molecule such as a fluorophore molecule or dye conjugated to the surface of the nanoparticle. In further embodiments, the fluorophore is contained within nanoparticle shell or in the core of the nanoparticle. In still further embodiments the nanoparticle itself is a fluorophore. In still further embodiments, the nanoparticle is a dye-doped silica nanoparticle. According to certain embodiments, a silica nanoparticle is conjugated to fluorescamine isothiocyanate (FITC). In further embodiments, rhodamine isothiocyanate In further embodiments, the fluorophore is selected from a group, including, but not limited to: fluorescein and fluorescein dyes (e.g., fluorescein isothiocyanine or FITC, naphthofluorescein, 4′,5′-dichloro-2′,7′-dimethoxy-fluorescein, 6-carboxyfluorescein or FAM), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red, Spectrum Green, cyanine dyes (e.g. Cy-3, Cy-5, Cy-3.5, Cy-5.5), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), HiLyte Fluor dyes, and eFluor dyes. In certain embodiments, a single fluorophore is used. In further embodiments multiple fluorophores are used. In still further embodiments, the multiple flourophores are excitable at different wavelengths such that they can be distinguished from one another.

Conjugation of antigens to nanoparticles is achieved through various means known in the art. For example, in certain embodiments, silica nanoparticles are prepared by the modified Stober method (Banerjee, et al. Tetrahedron Lett. 52:1878-1881(2011)). According to certain embodiments, nanoparticles are functionalized by the surface hydrolytic condensation of trialkoxysilylpropyl-derivatives of ammonia, polyethylene glycol, or another selected functional group that can facilitate linkage reactions with antigens or reporter molecules. According to further embodiments, electrostatic nanoparticle—protein coupling is used. According to still further embodiments a streptavidin-biotin system is used to facilitate coupling. One skilled in the art would appreciate that other approaches are possible.

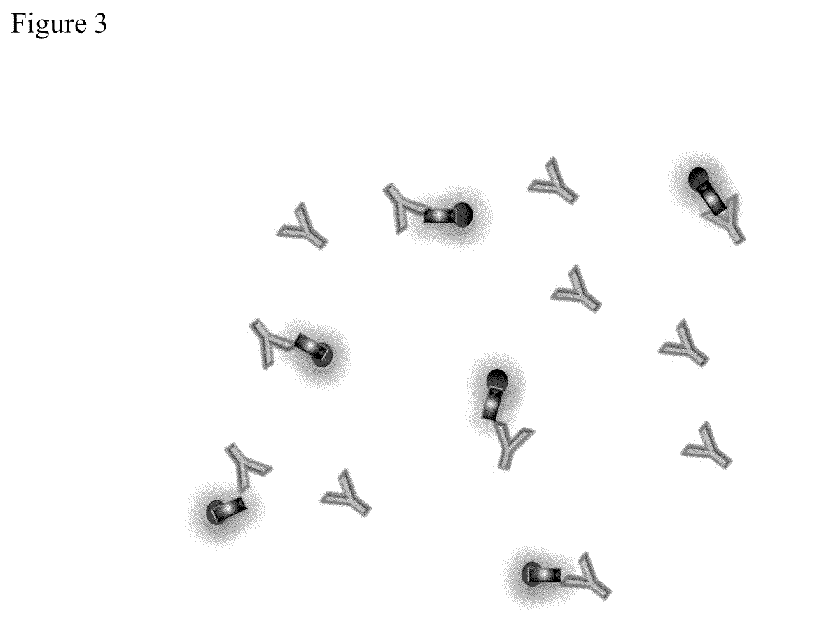

In certain embodiments, as shown in the schematic of FIG. 3, antigen-linked nanoparticles are contacted with serum to allow for binding of serum antibodies to the antigen of the antigen-linked nanoparticle. In certain embodiments, serum antibodies and antigen-linked nanoparticles are contacted through an incubation step. Parameters and conditions of the incubation step can vary according to the specific nanoparticle-antigen combination used.

### Cell Types

According to certain embodiments, the Fc receptor expressed by Fc receptor-expressing cells may include any Fc receptor subtype including but not limited to Fc receptors in the FcγR, FcαR, and FcεR classes. In certain aspects, Fc receptor-expressing cells include, but are not limited to, macrophages, neutrophils, natural killer cells, mast cells, B lymphocytes monocytes, polymorphonuclear leukocytes, any or all immortalized cell lines that express phagocytic activity or cyropreserved cells from animals or humans. In certain embodiments, the Fc receptor-expressing cells have endogenous expression of the Fc-receptor. In further embodiments Fc receptor expressing cells are stably transfected with an Fc-receptor transgene. In certain embodiments, the Fc receptor-expressing cells are primary cells. In still further embodiments, the Fc receptor-expressing cells are from commercially available cell lines, for example murine macrophage cell line J774A.1 available from ATCC, Manassas, Va.

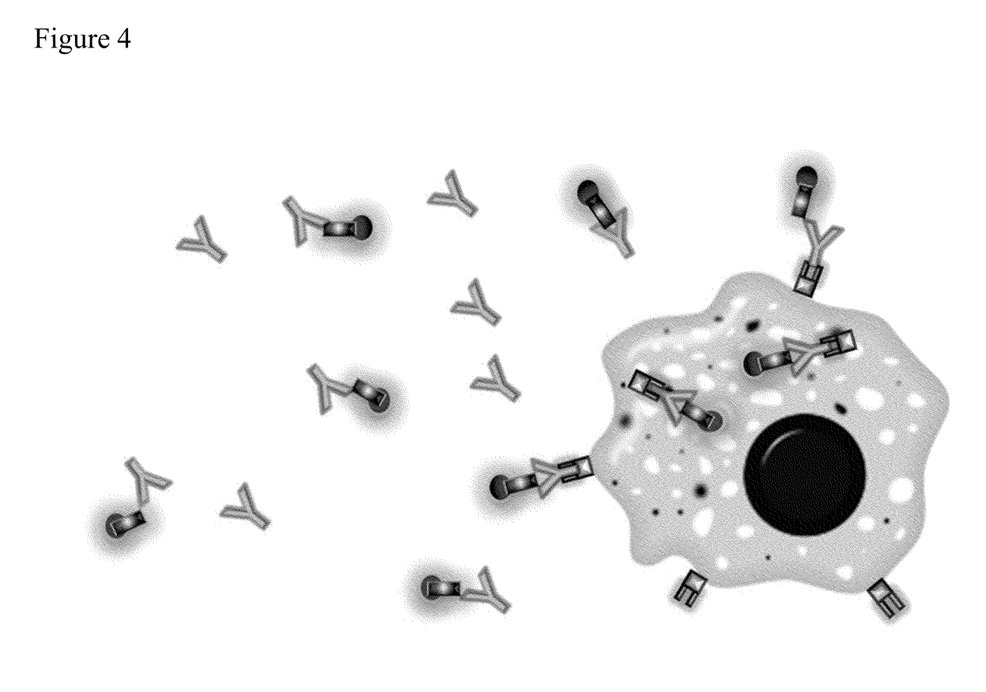

In certain embodiments, as shown in the schematic of FIGS. 4 & 5, following the step of contacting antigen-linked nanoparticles with serum, the nanoparticle/serum mixture is contacted with Fc receptor-expressing cells. According to certain embodiments, after an incubation period, cells are washed to remove antigen-linked nanoparticles that were not uptaken. In certain embodiments, the incubation time is about sixty minutes at a temperature of about 37° C. As will be appreciated by one skilled in the art, incubation conditions can vary.

### Methods of Measuring Uptake

In certain aspects the method relates to measuring amount antigen-linked nanoparticle uptake by the Fc receptor-expressing cells. The method of measuring the amount of antigen-linked nanoparticle uptake depends on the type of nanoparticle employed. For example when nanoparticles are labeled with fluorophore, uptake is measured by assessing fluorescence of the cells. Any technique known in the art for measuring fluorescence can be used. In certain embodiments, nanoparticle uptake is quantified by flow cytometery. According to certain embodiments, measuring the amount antigen-linked nanoparticle uptake by of the Fc receptor-expressing cells is accomplished through visualization by florescence microscopy In further embodiments, additional methods for visualizing and/or quantitating fluorescence associated with antibody:Fc receptor interactions are provided. Examples include but are not limited to: confocal microscopy and detection with instruments that specifically measure fluorescence including those that use a plate format (i.e. Synergy HT) or fluorescent microsphere immunoassay (i.e. Luminex system).

In certain embodiments, the method relates to the step of determining efficacy of the vaccine by comparing the level of antigen-linked nanoparticle uptake to a baseline level of uptake wherein a greater nanoparticle uptake compared to the baseline level of uptake is indicative of greater vaccine efficacy. According to certain embodiments, the baseline level of uptake is determined by measuring the level of uptake by Fc receptor-expressing cells of antigen-linked nanoparticles that have not been contacted by antibodies. In further embodiments baseline level of uptake is determine by measuring the level of uptake of antigen-linked nanoparticles that have been contacted by serum of an unvaccinated animal. In still further embodiments, baseline level of uptake is determined by measuring the level of uptake of antigen-linked nanoparticles that have been contacted by serum from animals vaccinated with a vaccine against an unrelated pathogen.

According to certain embodiments an individual subject's response to a vaccine is measured by collecting pre-vaccination serum from the subject and using said serum to establish a baseline for comparison with post-vaccination serum from the subject.

Accordingly, disclosed herein is method for determining the immunogenic effect of a vaccine on a subject comprising: obtain serum from the subject prior to a vaccination; obtaining serum the subject after a vaccination; contacting the pre-vaccination serum and post-vaccination serum with a plurality of antigen-linked nanoparticles, contacting the plurality of antigen-linked nanoparticles with a plurality of Fc receptor-expressing cells; measuring the amount of antigen-linked nanoparticle uptake by the Fc receptor-expressing cells; and determining the effect of the immunogenic effect of vaccine on the subject by comparing the amount of antigen-linked nanoparticle uptake induced by pre-vaccination serum with post vaccination serum wherein a greater immunogenic effect is indicated by a greater level of uptake by post-vaccination serum.

In certain embodiments, the methods disclosed herein are practiced as an assay. In further embodiments, the methods disclosed herein are practiced through use of a kit. In certain further embodiments, the method is a high throughput screen.

### EXAMPLES

### Synthesis of NanoFcR Nanoparticles

Fluorescein isiothiocyanate isomer I (90%, 5 mg, 1×10-5 mol) and of (3-aminopropyl)triethoxysilane (APTES, 3.1'10-4 mol) are stirred in 1 mL of absolute ethanol for 30 minutes, producing FITC-APTES conjugate. Concurrently, cyclohexane, Triton X-100, n-hexanol, and D.I. water was stirred together for 15 min, producing a water-in-oil micro-emulsion. To the micro-emulsion media, the FITC-APTES/ethanol solution (5×10-7 mol FITC-APTES, 1.5×10-5 mol APTES), tetraethyl orthosilicate (4.48×10-4 mol), and 14.5 M NH4OH (1.45'10-3 mol) was added. After stifling for 10 min, additional FITC-APTES/APTES/ethanol mixture, TEOS and NH4OH were added in proportions equal to their respective first portions. After 30 min of additional stirring, 3-(trihydroxysilyl) propyl methylphosphonate, monosodium salt, 42% in water (THPMP, 3.3×10-5 mol) was added and this final mixture is stirred for 24 h at room temperature. Ethanol is then added to disrupt the micro-emulsions. Nanoparticles were isolated, washed three times by repeated re-suspension in 1 mL of 95% ethanol and air-dried. The nanoparticle size was measured by TEM and the presence of surface amino-groups was confirmed by a qualitative ninhydrin test.

### Protein-Nanoparticle Conjugation

Stock solution of 1 mg/mL of succinic anhydride in N,N-dimethylformamide (DMF) is prepared. A 1 mL aliquot of this stock solution (1 mg of succinic anhydride, ×10-5 mol) was added to 5 mg of dry nanoparticles and then the mixture was sonically agitated to achieve suspension of the nanoparticles. The mixture was stirred for 30 min, producing carboxylic acid-functionalized nanoparticles. The nanoparticles were isolated by centrifugation and decantation, with the precipitate washed three times with 95% ethanol. A qualitative ninhydrin test of the resulting nanoparticles is used to confirm the lack of amine functionality. The resulting carboxylic acid groups are activated by subsequent reaction with 2 mg of 1-ethyl-3-(3-dimethylaminopropyl) carbodiimide (EDC, 1.2×10-5 mol) and 1 mg of N-hydroxysulfosuccinimide (sulfo-NHS, 5×10-6 mol). The nanoparticles are then isolated by centrifugation and washed with 0.1 M, pH 7.4 PBS. Finally, 300 μL of a 3-mg/mL CA09 recombinant HA protein in 0.1 M, pH 7.4 PBS (1 mg, MW=44 kDa, 2.3×10-8 mol) is added to the nanoparticles, previously suspended in 1 mL of 0.1 M, pH 7.4 PBS and the reaction mixture is shaken for 5 hrs. Protein-nanoparticle conjugates are isolated by centrifugation and washed twice by repeated re-suspension in PBS.

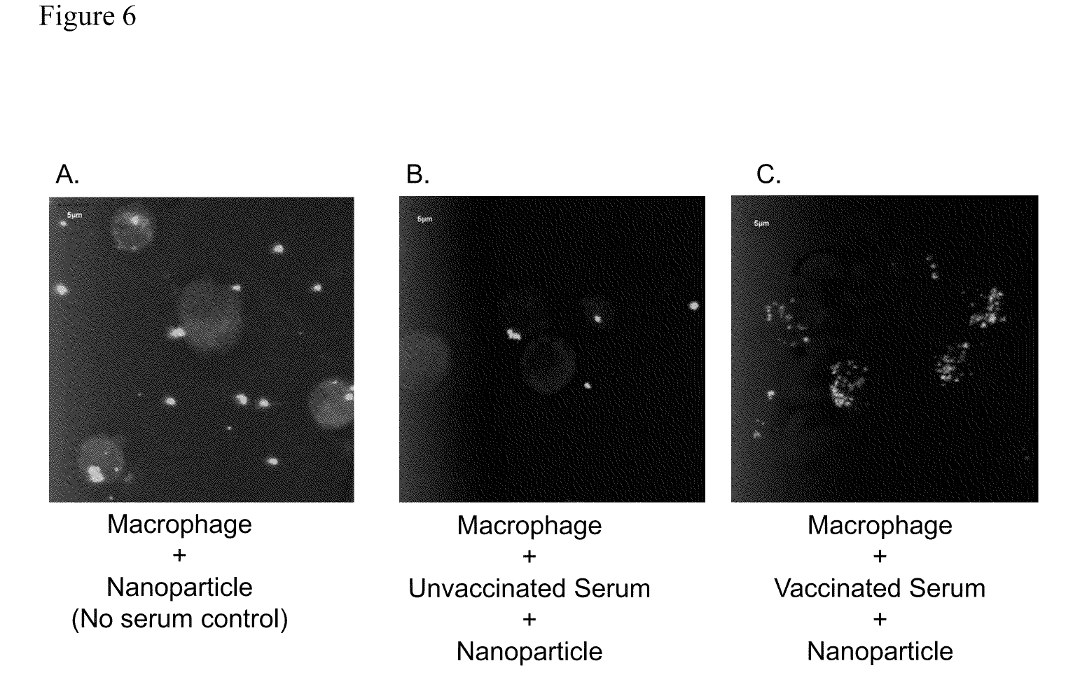

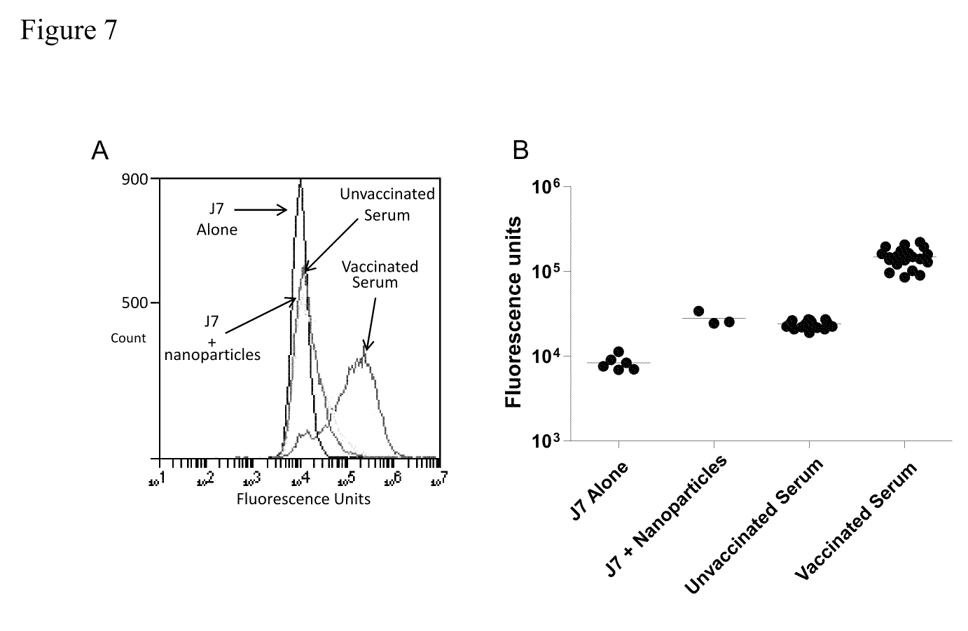

Successful cellular imaging is achieved by treatment of cells with 0.5 mL of a 5-mg/mL suspension of nanoparticles (3×10-3 nmol of particles). For comparison to the protein-nanoparticle conjugates, the control experiment used carboxylic acid-functionalized nanoparticles (no sulfo-NHS activation or protein conjugation). FIG. 7 shows flow cytometry results and FIG. 6 shows confocal images of macrophages mixed with CA09 HA-conjugated nanoparticles in the presence of serum. Here 3 μL of FITC labeled HA-conjugated nanoparticles in PBS+0.2% BSA were combined with 1 μL of indicated pooled or individual murine sera and incubated for 60 minutes at 37° C. with shaking. One million J774A.1 BALB/c murine macrophage (ATCC, Manassas, Va.) were then added and the reactions incubated for 60 minutes at 37° C. with shaking and followed by washes. Sera induced uptake by macrophage was analyzed using an Accuri C6 flow cytometer (Accuri Cytometers Ltd., Ann Arbor, Mich.) and accompanying CFlow Plus software (Accuri).

For confocal imaging, cell samples were dried on slides and submerged in ethanol and xylene. Slides were prepared with cytoseal 60 and coverslips imaged using an Olympus Fluoview 1000 laser-scanning confocal microscope (Olympus America, Inc., Center Valley, Pa.) from which z-stack optical sections were obtained. Samples were scanned using a 60×1.4 numerical aperture oil-immersion objective and 488-nm argon laser. Visualization of this uptake is demonstrated in FIG. 6.

FIG. 7 shows that flow cytometry allows for quantitation of macrophage uptake of fluorescent nanoparticles in the presence of individual serum samples. The histogram (A) demonstrates individual fluorescence peaks for a single representative from each group, while (B) demonstrates the fluorescence units for multiple serum samples within each group. The groups indicated are J7 alone (n=6), J7+nanoparticles (n=3), J7+nanoparticles+sera from vaccine vehicle (n=20), and J7+nanoparticles+serum from vaccinated animals (n=20).

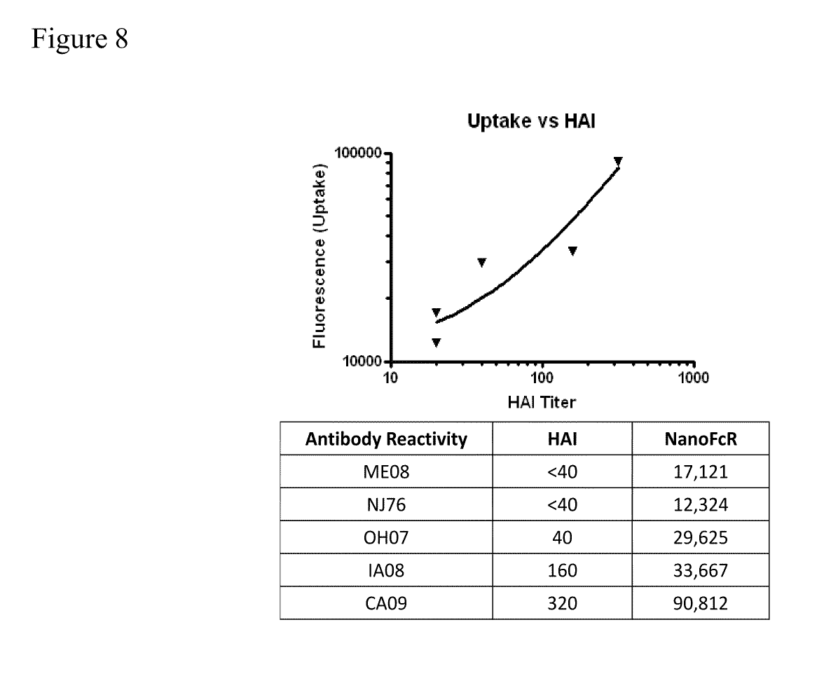

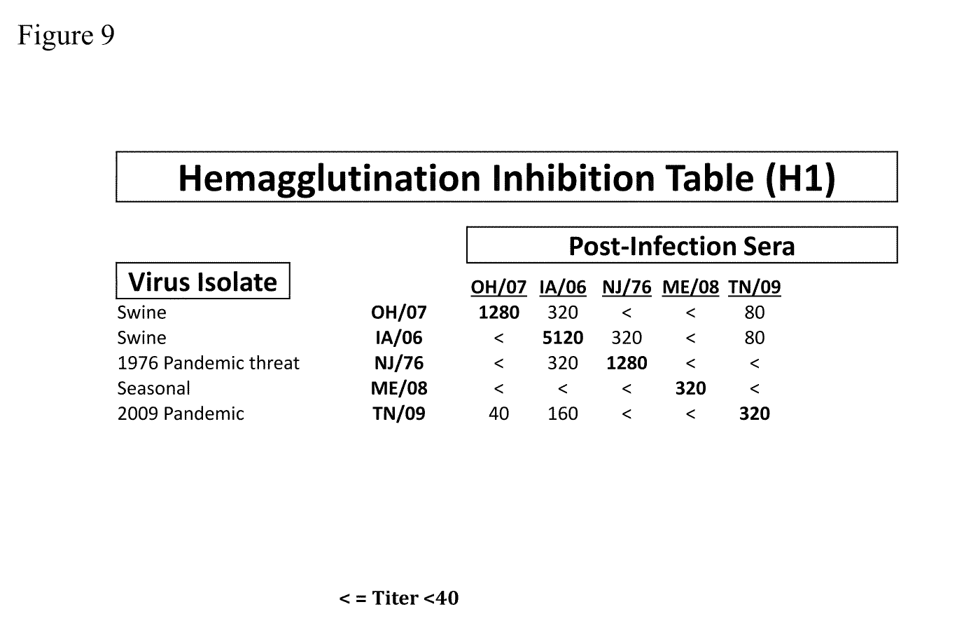

FIGS. 8 & 9 shows data comparing serum reactivity toward the influenza virus CA09 HA using HAI and methods of the present invention (NanoFcR) (FIG. 9). Results presented compare serum performance within the uptake assay with performance in the HAI assay (raw data presented in table for HAI titer and NanoFcR Mean Fluorescence Intensity). Using the HAI assay, reactivity of the individual serum samples with their respective HAs was as follows: ME08=320, NJ76=1280, OH07=1280, IA06=5120, and CA09=320.

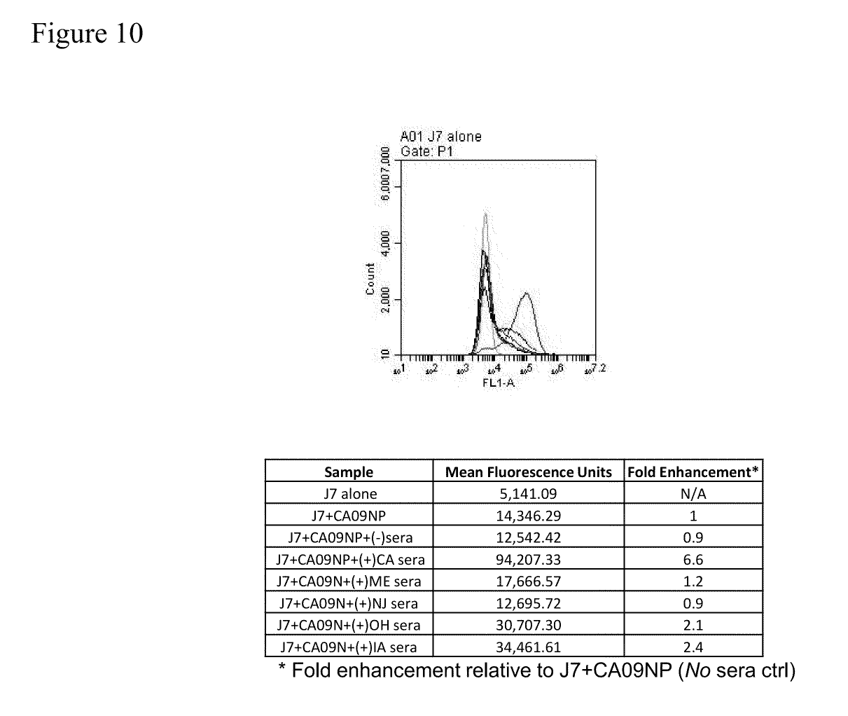

FIG. 10 shows data demonstrating nanoparticle uptake specificity. Specific and non-specific murine sera tested for the ability to induce the opsonophagocytosis of CA09 hemagglutanin (HA) conjugated nanoparticles by J774A.1 (Balb/c) macrophage.

Although the present invention has been described with reference to preferred embodiments, persons skilled in the art will recognize that changes may be made in form and detail without departing from the spirit and scope of the invention.

1. A method for determining the efficacy of a vaccine comprising:
a. providing serum from an animal inoculated with a vaccine;
b. providing a plurality of antigen-linked nanoparticles;
c. contacting the serum with the plurality of antigen linked nanoparticles;
d. contacting the serum and the plurality of antigen linked nanoparticles with a plurality of Fc receptor-expressing cells;
e. measuring amount antigen-linked nanoparticle uptake by of the Fc receptor-expressing cells;
f. determining efficacy of the vaccine by comparing the level of antigen-linked nanoparticle uptake to a baseline level of uptake wherein a greater nanoparticle uptake compared to the baseline level of uptake is indicative of greater vaccine efficacy.

2. The method of claim 1 wherein the baseline level of uptake is determined by contacting a plurality of serum-free antigen-link nanoparticles with a plurality of Fc receptor-expressing cells and measuring uptake by the Fc receptor-expressing cells.

3. The method of claim 1 wherein the baseline level of uptake is determine by providing serum from an unvaccinated animal and performing steps b through e with said serum.

4. The method of claim 1 wherein the antigen-linked nanoparticle further comprises a fluorophore.

5. The method of claim 4 further wherein measuring amount antigen-linked nanoparticle uptake by of the Fc receptor-expressing cells is done by measuring fluorescence of the cell.

6. The method of claim 5 wherein florescence is measured by flow cytometry.

7. The method of claim 1 wherein the antigen is associated with a pathogen to which the vaccine is directed.

8. The method of claim 7 wherein the pathogen is viral, bacterial, or fungal.

9. The method of claim 1 wherein the antigen is a recombinant protein, recombinant peptide, native protein or native peptide, chemically synthesized peptide, native carbohydrate or chemically synthesized carbohydrate.

10. The method of claim 1 wherein said fluorophore is FITC.

11. The method of claim 1 wherein said antigen is a recombinant protein.

12. The method of claim 1 wherein said cells are macrophages.

13. The method of claim 1 wherein said nanoparticles are silica nanoparticles.

14. The method of claim 1, wherein the cells are neutrophils.

15. The method of claim 1, wherein the cells are natural killer cells.

16. The method of claim 1, wherein the cells are mast cells.

17. The method of claim 1, wherein the cells are B lymphocytes.

18. A method for determining the immunogenic effect of a vaccine on a subject comprising:
a. obtain serum from the subject prior to a vaccination;
b. obtaining serum the subject after a vaccination;
c. contacting the pre-vaccination serum and post-vaccination serum with a plurality of antigen-linked nanoparticles,
d. contacting the plurality of antigen-linked nanoparticles with a plurality of Fc receptor-expressing cells;
e. measuring the amount of antigen-linked nanoparticle uptake by the Fc receptor-expressing cells; and
f. determining the effect of the immunogenic effect of vaccine on the subject by comparing the amount of antigen-linked nanoparticle uptake induced by pre-vaccination serum with post vaccination serum wherein a greater immunogenic effect is indicated by a greater level of uptake by post-vaccination serum.

19. (canceled)

20. (canceled)
